(19) United States
(12) Patent Application Publication
(10) Pub. No.: US 2007/0005005 A1
(43) Pub. Date: Jan. 4, 2007

(54) Method and apparatus for dosing solids

(76) Inventor: Fang Nai Wang, Long Island City, NY (US)

Correspondence Address: LUCAS & MERCANTI, LLP, 475 PARK AVENUE SOUTH, 15TH FLOOR, NEW YORK, NY, 10016, US

(21) Appl. No.: 10/570,296

(22) PCT Filed: Aug. 27, 2004
(86) PCT No.: PCT/US04/28097

§ 371 Date: Feb. 28, 2006

Related U.S. Application Data
(60) Provisional application No. 60/498,795, filed on Aug. 29, 2003.

(51) Int. Cl. A61M 31/00

(52) U.S. Cl. 604/60

(57) ABSTRACT

An apparatus for selectively dosing a solid is provided and includes (1) a flexible guide tube that include a first end and an opposing second end and has a bore formed therethrough from the first end to the second end; (2) a dispensing tip disposed at the first end of the guide tube and including a bore formed completely therethrough and defining a reservoir fore holding one or more dosing solids until actuation of the apparatus; and (3) a stylet that is dimensioned so as to be slideably received within through the bore of the flexible guide tube, the stylet having a first end and an opposing second end. With the first end of the stylet being for contact with one or more dosing solids to cause discharge thereof through the dispensing tip.

### CROSS-REFERENCE TO RELATED APPLICATION

This application claims the benefit of U.S. patent application Ser. No. 60/498,795, filed Aug. 29, 2003, which is hereby incorporated by reference in its entirety.

### TECHNICAL FIELD

The present invention relates to a method and apparatus for dosing solids, especially small solids, and the solids used for such a method. In addition, the present invention relates to dosing of solids to animals, including mammals, especially rodents and humans, birds, fish, reptiles, and insects.

### BACKGROUND

Animal testing has long been a mainstay in research, especially in pharmaceutical research. Dosing of animals by the oral route to test a preparation has been an important aspect of the research. Since many preparations will act differently physiologically based on the route of administration, it has been important to test oral drugs by oral administration. Scientists have known that especially in small animals like rats, dosing of a solid poses many problems. To be able to get accurate results, the preparation must be delivered to the proper site, usually the esophagus and/or stomach. So to get to the stomach, the solid dosage form must travel from the mouth or the nose, usually the mouth, down the esophagus and into the esophagus and/or stomach. Small animals are not likely to voluntarily take the solid as may be needed for the testing. Therefore, the scientist must find a way to dose the solid. Dosing apparatus for this use currently on the market have the drawback of either being too rigid and possibly fatally perforating the animals esophagus, or too flexible and not being able deliver the solid to the correct location. Additionally, the dosage form must be small enough to pass into the animal's stomach. Torpac Inc. of Fairfield, N.J. has a size 9 capsule dosing kit for dosing rodents. The kit consists of a rigid metal dosing syringe designed to be used with a size 9 capsule and size 9 capsule. The disadvantages of such a method, including perforating or otherwise damaging the esophagus, as well as that the sharp, wire plunger on the Torpac device could poke a hole or fracture the solid dosage form. In addition, the Torpac device has a number of limitations associated with the size of medications that can be received and held therein and more particularly, the device has an integral tip of a pre-determined size that can only accommodate certain size capsules, etc. Thus, the user may need a number of different Torpac devices to cover the range of medications that are to be dispersed to the mammal, etc.

### SUMMARY

The present invention relates to an apparatus capable of delivering solid dosage forms to the esophagus and/or stomach of an animals, including mammals, especially rodents (including rats, guinea pigs and mice), dogs, cats, sheep, cows, horses and humans, birds, fish, reptiles, and insects, with minimal risk of physical damage to the esophagus, gastro-intestinal tract by the apparatus.

More specifically, the present invention is a dosing apparatus that includes a flexible dosing guide tube with a flexible dosage dispensing tip member projecting parallel with the plane of the dosing guide tube at one end of the dosing guide tube. The dispensing tip member is capable of holding at least one solid dosage form. Coupled at the other end of the dosing guide tube is optionally another body member similar in shape to a female luer and projecting parallel with the plane of the dosing guide tube. The dosing apparatus further comprises a plunger or stylet that is positioned down the longitudinal center of the inside of the dosing guide tube. A first end of the stylet preferably includes a tip member integral with the first end and projecting parallel with the longitudinal plane of the stylet. The stylet tip member is used to push the solid dosage form out of the dispensing tip of the dosing tube. Optionally at a second end of the stylet is a finger support which may be round, curved or flat. Optionally at a defined distance from the stylet tip member along the length of the stylet is a mechanical stop to stop the advancement of the stylet too far beyond the flexible dispensing tip member of the dosing guide tube.

The apparatus preferably further includes a stop mechanism that selectively limits the axial travel of the stylet within the guide tube. In one embodiment, the stop mechanism is in the form of a lock and key arrangement with a stop feature formed along the length of the stylet acting as the key and the stop mechanism being part of the additional body member coupled to the guide tube and acting as a lock that permits axial travel of the stylet only when the lock and key are in proper registration.

The invention further relates to a method of dosing a solid dosage form to an animal. The method comprising attaching a solid dosage form to the above dosing apparatus that includes the flexible dosing tube coupled to the flexible dosage dispensing tip member and a stylet. The dosing apparatus is inserted into the animal's pharynx and esophagus and then the stylet is pushed or axially directed into the dosing guide tube to release the solid dosage form.

### BRIEF DESCRIPTION OF THE DRAWING FIGURES

One or more embodiments of the invention and of making and using the invention, as well as the best mode contemplated of carrying out the invention, are described in detail below, by way of example, with reference to the accompanying drawings, in which:

FIG. 1 is an exploded perspective view of a dosing device according to one exemplary embodiment;

FIG. 2A is a cross-sectional view taken along the line 2-2 of FIG. 1 illustrating a dosage dispensing tip member that contains a dosing solid form and is mated with a guide tube;

FIG. 2B is a cross-sectional view taken along the line 2-2 of FIG. 1 with a stylet inserted into the guide tube and the dosage dispensing tip member to dislodge the dosing solid form;

FIG. 3 is a cross-sectional view taken along the line 3-3 of FIG. 1 with the guide tube shown exploded from the dosage dispensing tip member;

FIG. 4A is a cross-sectional view taken along the line 4-4 of FIG. 1 showing a dosage dispensing tip member, according to a second embodiment in which multiple dosing solid forms can be stored, mated to the guide tube;

FIG. 4B is a cross-sectional view taken along the line 4-4 of FIG. 1 with the stylet disposed into the dosage dispensing tip member to dislodge the multiple dosing solid forms;

FIG. 5A is a cross-sectional view taken along the line 5-5 of FIG. 1 showing a dosage dispensing tip member, according to a third embodiment in which multiple dosing solid forms can be stored, mated to the guide tube;

FIG. 5B is a cross-sectional view taken along the line 5-5 of FIG. 1 with the stylet disposed into the dosage dispensing tip member to dislodge the multiple dosing solid forms;

FIG. 6 is cross-sectional view taken along the line 6-6 of FIG. 5B;

FIG. 7A is a cross-sectional view taken along the line 7-7 of FIG. 1 showing an end body member mated to one end of the guide and including a stop mechanism according to a first embodiment for limiting the degree of travel of the stylet;

FIG. 7B is a cross-sectional view taken along the line 7-7 of FIG. 1 showing deformation of the stop mechanism;

FIG. 8 is a cross-sectional view taken along the line 8-8 of FIG. 7A;

FIG. 9A is a cross-sectional view of the end body member mated to one end of the guide and including a stop mechanism according to a second embodiment for limiting the degree of travel of the stylet;

FIG. 9B is a cross-sectional view showing deformation of the stop mechanism of FIG. 9A;

FIG. 10 is a cross-sectional view taken along the line 10-10 of FIG. 9A;

FIG. 11A is a cross-sectional view of the end body member mated to one end of the guide and including a stop mechanism according to a third embodiment for limiting the degree of travel of the stylet;

FIG. 11B is a cross-sectional view showing deformation of the stop mechanism of FIG. 11A;

FIG. 12 is a cross-sectional view taken along the line 12-12 of FIG. 11A;

FIG. 13 is a cross-sectional view taken along the line 13-13 of FIG. 11A;

FIG. 14A is a cross-sectional view of the end body member mated to one end of the guide and including a stop mechanism according to a fourth embodiment for limiting the degree of travel of the stylet;

FIG. 14B is a cross-sectional view showing deformation of the stop mechanism of FIG. 14A;

FIG. 15 is a cross-sectional view taken along the line 15-15 of FIG. 14B;

FIG. 16 is a graph showing the percent change in glucose levels from a baseline during a first solid dose application; and

FIG. 17 is a graph showing the percent change in glucose levels from a baseline during a second solid dose application.

### DETAILED DESCRIPTION OF PREFERRED EMBODIMENTS

Ranges of sizes for the inventive dosing tube and solid dosage form vary with the size and weight of the animal to be dosed, including but not limited to the width and length of the mouth and esophagus. The below values are illustrative and relate to the dosing of a rat of about 230 g to about 250 g.

Additionally, unless expressly stated to the contrary, all values, sizes, measures and the like are approximate.

In addition, the present invention relates to dosing of solids to animals, including mammals especially rodents and humans, birds, fish, reptiles, and insects.

For the purposes of this application, flexible is defined as a level of bendability, that is higher, preferably significantly higher, than that of a dosing element that is made of metal, such as Torpac's stainless steel dosing syringe or the like. For the purposes of this application, a solid dosage form can be a tablet or a capsule. A tablet can be in the form of a caplet, oval, a bead, a disc or elliptical. A capsule can be filled with a liquid and/or solid, including a semisolid or gel.

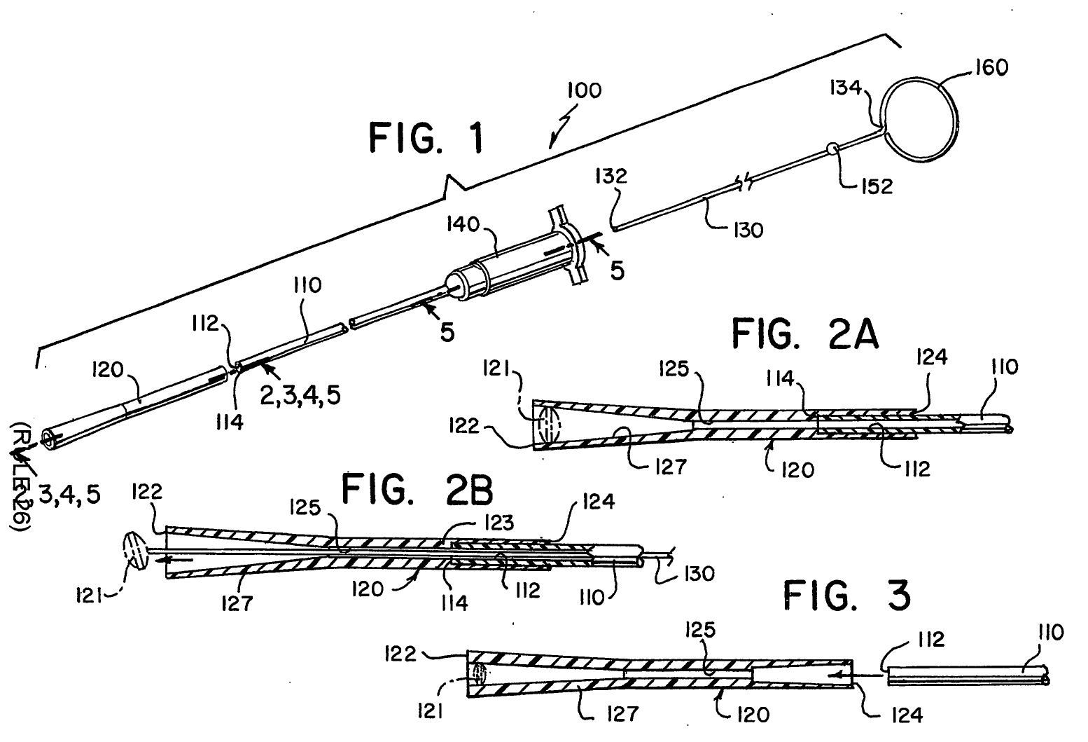

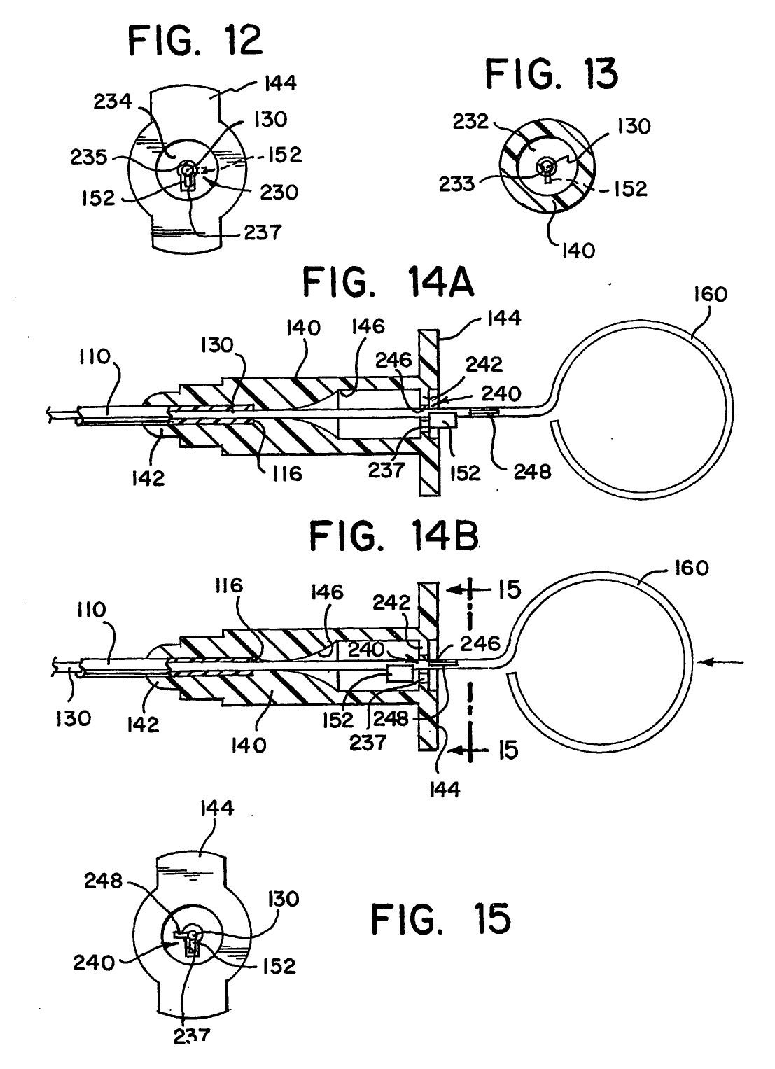

FIGS. 1-15 illustrate a dosing device 100, according to a first embodiment, that includes a flexible dosing guide member 110 of sufficient length and circumference to dose an animal in question, e.g., a rodent, such as a rat, using conventional dosing techniques. The dosing device 100 according to the first embodiment also includes a plunger or stylet 130 that is slideably received within a bore 112 formed through the dosing guide member 110 from a first end 114 to a second end 116, as well as a dosage dispensing tip member 120 that is coupled to the first end 114 of the dosing guide member 110 and is configured to receive an hold medication to be dispensed as a dose to the animal, as will be described in greater detail hereinafter.

The dosing guide member 110 is constructed such that the bore 112 is sized to slideably receive the stylet 130 and it must be narrow enough to fit down the esophagus of the animal to be dosed without damaging any anatomical structure, but large enough for practical dosing of the animal. In the illustrated embodiment and according to one preferred design, the dosing guide member 110 has a generally cylindrical body and thus takes the form of a dosing guide tube that has an annular bore 112 extending completely therethrough.

In addition, the dosing guide member 110 must be of sufficient length to reach the desired delivery point (i.e., base of the esophagus or stomach). For example, for dosing of rats (weighing about 230 g to about 250 g), the dosing tube should be of a length 12 between about 80 mm to about 150 mm, preferably about 100 mm, which is of a length similar to those an existing product sold by Instech Laboratories, Inc. of Plymouth Meeting, Pa. under part number FTP-15-100. According to one exemplary embodiment, the dosing guide member 110 has an outer diameter from about 1.0 mm to about 2.8 mm, preferably about 1.8 mm, while the inner diameter thereof is from about 0.5 mm to about 1.8 mm, preferably about 1.2 mm. However, it will be appreciated that these dimensions are merely illustrative and not limiting of the present invention and the dosing guide member 110 can have a range of dimensions so long as the stylet 130 is slideably received therein and preferably is completely removable therefrom.

As described in more detail below, one of the properties of the dosing guide member 110 is that the body thereof is flexible in nature so as to permit dosing device 100 to travel more easily within and down the esophagus of the animal during a dosing operation. As previously mentioned, conventional dosing devices do not incorporate the level of flexibility as the present device 100 and thus suffer from a number of disadvantages including the greatly increased risk that the device 100, and more particularly, the distal end of the dosing guide tube 110, will perforate or otherwise damage the esophagus of the animal. Since the bodies of conventional dosing devices are formed of metal, such as stainless steel, they are extremely rigid as opposed to being flexible.

At the first end 114 of the dosing guide tube 110 is the dosage dispensing tip member 120 that is likewise flexible in nature and lies along the same longitudinal axis as the dosing guide tube 110. The dosage dispensing tip member 120 is constructed and configured such that it is capable of receiving and holding a dosing solid 121 (e.g., a capsule or tablet) at a first end 122 thereof until the dosing device 100 is positioned to a target site at which time, the dosing device 100 is actuated and the longitudinal axial travel of the stylet 130 within the bore 112 of the guide tube 110 causes displacement of the dosing solid 121 held at the end 122.

According to one embodiment that is illustrated in the figures, the dosage dispensing tip member 120 has a generally cylindrical body 123 that has a bore 125 formed therethrough from the first end 122 to an opposing second end 124. Similar to the dosing guide tube 110, the dosage dispensing tip member 120 has a tubular construction. Preferably, the cylindrical body 123, as well as the bore 125, does not have a uniform construction in that each has a tapered construction. More specifically, the dosage dispensing tip member 120 is constructed such that the first end 122 is generally of a frustoconical design, defined by an annular wall that tapers inwardly toward the second end 124 until a point between the ends 122, 124 where the annular wall assumes a uniform diameter along its length to the second end 124. In other words, the greatest diameter of the dosage dispensing tip member 120 is at the first end 122. Similarly, the bore 125 is not of a uniform diameter but rather it is of varying diameter and itself has a tapered construction in that the bore 125 has an enlarged section 127 at the first end 122 where the diameter of the bore 125 is at its greatest before the bore 125 tapers inwardly to a point where the diameter remains constant. This enlarged section 127 of the bore 125 defines a reservoir that can receive and hold one or more dosing solids 121.

In one exemplary embodiment, the dosage dispensing tip member 120 has an outer diameter of from about 2 mm to about 6 mm, preferably about 4 mm at its widest part (first end 122) and an inner diameter, defined as the diameter of the bore 125, of from about 1 mm to about 5 mm, preferably about 3 mm at its widest part (enlarged section 127). However, these dimensions are merely exemplary and illustrative in nature and do not serve to limit the present invention since the dosage dispensing tip member 120 can be formed to have any number of different dimensions depending upon the particle application. In particular, the dimensions of the enlarged section 127 will vary depending upon the size of the dosing solid 121 that is received therein.

When the dosage dispensing tip member 120 is coupled to the first end 114 of the dosing guide tube 110, the bores 112, 125 are axially aligned with one another to permit passage of the stylus 130 from the dosing guide tube 110 to the medication dispensing tip member 120. It will be appreciated that the dosage dispensing tip member 120 can be attached to the dosing guide tube 110 by any means known in the art such as by using an adhesive, welding, crimping or by a frictional fit, or be a molded part of the dosing guide tube 110. In other words, the dosing guide tube 110 and the dosage dispensing tip member 120 can be two separate members which are either non-detachably secured to one another or are detachably secured to one another, as by a releasable frictional fit in which one of the members is slidably received in the bore of the other member, e.g., the dosage dispensing tip member 120 can function as a sleeve which is slid over a length of the dosing guide tube 110 that includes the first end 114. The reverse can be true as the dosage dispensing tip member 120 can be received within the bore 112 of the dosing guide tube 110.

It will be appreciated that in the embodiment where the dosage dispensing tip member 120 is frictionally fit to the dosing guide tube 110, the dosage dispensing tip member 120 is capable of being interchanged with the dosing guide tube 110. This is particularly advantageous since the dosing device 100 can marketed as a kit that includes not only the basic components (guide tube 110 and stylet 130) but also contains a number of interchangeable dosage dispensing tip members 120 that can be individually selected, in view of the intended application, so as to alter the dispensing characteristics of the device 100. More specifically, the kit can include dosage dispensing tip members 120 that have reservoirs 127 of different dimensions so as to permit different sized and different shaped dosage solids 121 to be received and stored therein. For example, when the intended use is to administer a capsule, the reservoir 127 can have a complementary shape thereto, such as an oval shape, while the remaining length of the bore 125 can have a circular cross-section. When a tablet is to be administered, the reservoir 127 can simply be an enlarged diameter circular cross-section compared to the remaining length of the circular cross-section bore 125. In addition and as described in more detail below, the reservoir 127 can be constructed to hold more than one dosing solid 121 in such a way that the dosing solids 121 are separated from one another, thereby permitting separate controlled administrations.

By supplying a number of interchangeable dosage dispensing tip members 120, the user does not have to buy multiple dosing devices 100 as is typically the case with the conventional devices but rather, the user can simply purchase different interchangeable dosage dispensing tip members 120 and when one is needed, the existing is slidably removed from end 114 of the guide tube 110 and the new dosage dispensing tip member 120 is then simply inserted about and slidably travels over the outer surface of the guide tube 110 until it reaches an engaged position. Having interchangeable dosage dispensing tip members 120 is a significant improvement over the conventional dosing devices which only offered one type of tip member for each device 100.

While, the dosage dispensing tip member 120 can be formed in any number of different manners, e.g., by being formed in-situ with the guide tube 110 during a molding process, in one embodiment, the dosage dispensing tip member 120 is made by starting with a Nelaton Catheter RR size 8 French 25 such as is sold by Rusch, Inc. of Duluth, Ga. under product number 402140080. The dosage dispensing tip member 120 is made by cutting a first section across a funneled end of the catheter at an area that has the desired diameter. A second cut is made along catheter such that the dosage dispensing tip member 120, defined as the portion remaining between the first and second cuts, is of sufficient length to be placed over and retained on first end 114 of dosing guide tube 110 at the second end 124 thereof, while the first end 122 is adapted to retain one or more dosage solids 121.

Coupled to the second end 116 of the dosing guide tube 110 is optionally a complementary body member 140 that can serve to limit the degree of travel of the stylet 130 with respect to the dosing guide tube 110. The body member 140 is generally a hollow member that includes a first end 142 that is coupled to the second end 116 of the dosing guide tube 110 and an opposing second end 144 that faces away from the dosing guide tube 110. In the illustrated embodiment, the body member 140 has a shape that is similar to a female luer that has a non-uniform diameter along its length from the first end 142 to the second end 144. It will be appreciated that when the body member 140 is coupled to the dosing guide tube 110, a bore 146 formed through the body member 140 from end 142 to end 144 is axially aligned with the bore 112 formed in the dosing guide tube 110.

In the illustrated embodiment, the outer surface of the body member 140 has a stepped construction in that one or more annular shoulders are formed along its length and preferably an end flange is provided to permit the user to easily grasp and hold the device 100 while the user pushes the stylet 160 such that it extends into the guide tube 110. However, the first end 142 thereof preferably has the smaller diameter as compared to the second end 144 thereof, which can typically include an annular flange 145 formed at the second end 144. The bore 146 formed through the body member 140 can either have a constant diameter or it can have a diameter that varies by the different sections of the body member 140. In any event, the bore 146 has a diameter that can accommodate the stylet 130 and preferably, the diameter of the bore 146 is significantly greater than the outer diameter of the stylet 130 along the entire length of the body member 140. In one embodiment, an outer diameter of the body member 140 at its widest part is from about 5 mm to about 9 mm, preferably about 7 mm and the body member 140 has an inner diameter of from about 2 mm to about 6 mm, preferably about 4 mm, with the body member 140 projecting parallel with the plane of the dosing guide tube 110. As with the dosage dispensing tip member 120, if present, the body member 140 can be attached to the second end 116 of the dosing guide tube 110 by any means known in the art, such as by using an adhesive, by welding or by a frictional fit or other type of mechanical fit, or alternatively, the body member 140 can simply be molded in-situ with the dosing guide tube 110.

The dosing guide tube 110 and all the components coupled thereto, namely, the dosage dispensing tip member 120, and the optional body member 140, can be formed of any one of or a combination of suitable natural or man-made material, such as medical grade silicone, rubber, polyethylene, nylon, plastics, such as polypropylene, or metal, or any combination therefrom. Alternatively and as previously-mentioned, the dosing guide tube 110 and components 120, 140 can be formed as a single molded piece of material or made of multiple sections held together by any means known in the art such as the use of an adhesive material, by welding or by a mechanism fit, such as a frictional fit.

As mentioned above, the dosing device 100 includes the stylet or plunger 130 that is slideably received within the bore 112 of the dosing guide tube 110. At least the length of the stylet 130 that is received within the bore 112 has an outer diameter that is less than the inner diameter of the bore 112, and in one embodiment of the present invention, the stylet 130 has an outer diameter of about 0.5 mm to about 2 mm, preferably about 1 mm and the stylet 130 has a length of about 100 mm to about 300 mm, preferably about 180 mm. In one embodiment of the present invention, the stylet 130 can be of the type used in the Torpac Inc. of Fairfield, N.J. “Size 9 Dosing Kits—Dosing Tube”. A first end 132 of the stylet 130 comprises a tip member 150 (FIG. 5B) that is integral with the first end 132 and projects parallel with the plane of the stylet 130. The tip member 150 has a diameter of about 1 mm to about 6 mm, preferably about 2 mm. In one embodiment of the present invention, the tip member 150 is made by taking a pipette tip, similar to one sold by Rainin Instruments of Woburn, Mass. under catalog number RC-L20ML, and making the below described modification. A distal end section, (e.g., about a 5 mm section) is cut off a narrowed end of the pipette tip and its narrow end is placed first onto the first end 132. A void in the center of the pipette tip can be filled with anything known in the art, such as wax, plastic, glue, or a section of insulated wire, such as is sold by Radio Shack of Fort Worth, Tex. under catalog number 278-501. The stylet 130 tip member is used to push the solid dosage unit out of the tip member 150 of the dosing guide tube 110.

Optionally, at a second end 134 of the stylet 130 is a finger support 160, which can be in any shape, such as round, flat, or curved. The finger support 160 is intended to receive a finger of the user so as to allow the user to grip and manipulate the stylet 130. For example, a finger is inserted through the finger support 160 and then the user can slideably move the stylet 130 in the bore 112 so as to either extend or retract the stylet 130 relative to the dosing guide tube 110.

Optionally at a defined distance from the tip member 150 and the finger support 160, along the length of the stylet 130, is an interference element 152 that serves to limit and restrict advancement of the stylet 130 relative to the dosing guide tube 110 and more particularly, the interference element 152 serves to restrict the longitudinal travel of the stylet 130 within the aligned bores so as to stop the advancement of the stylet 130 too far beyond the flexible expansion of the dosing guide tube 110. In one embodiment of the invention, when stylus 130 is advanced sufficiently through dosing guide tube 110, interference element 152 will engage and rest in the body member 140 of dosing guide tube 110 preventing further motion in that direction as described below in greater detail.

The stylet 130 and all of it components (tip member 150, optional finger support 160 and optional interference element 152) can be formed of any one of or a combination of suitable natural or man-made material, such as medical grade silicone, rubber, polyethylene, nylon, plastic, such as polypropylene, or a metal, such as stainless steel, or any combination therefrom. For example, the stylet 130 and its components can be a single molded piece of plastic material or made of multiple sections held together by any means known in the art such as adhesive, welding, crimping or friction. In addition, the stylet 130 and the finger support 160 can be formed of stainless steel, while the tip member 150 can be formed of a plastic material. The interference element 152 is merely a member or structure that creates an interference with the body member 140, or the guide tube 110 if the body member 140 is not present, and therefore, the precise shape and size of the interference element 152 is highly variable. For example, the interference element 152 can be an annular ring or collar that is formed around the circumference of the stylet 130 or it can be an interference tab or protrusion that projects locally from the stylet 130 as shown in the figures.

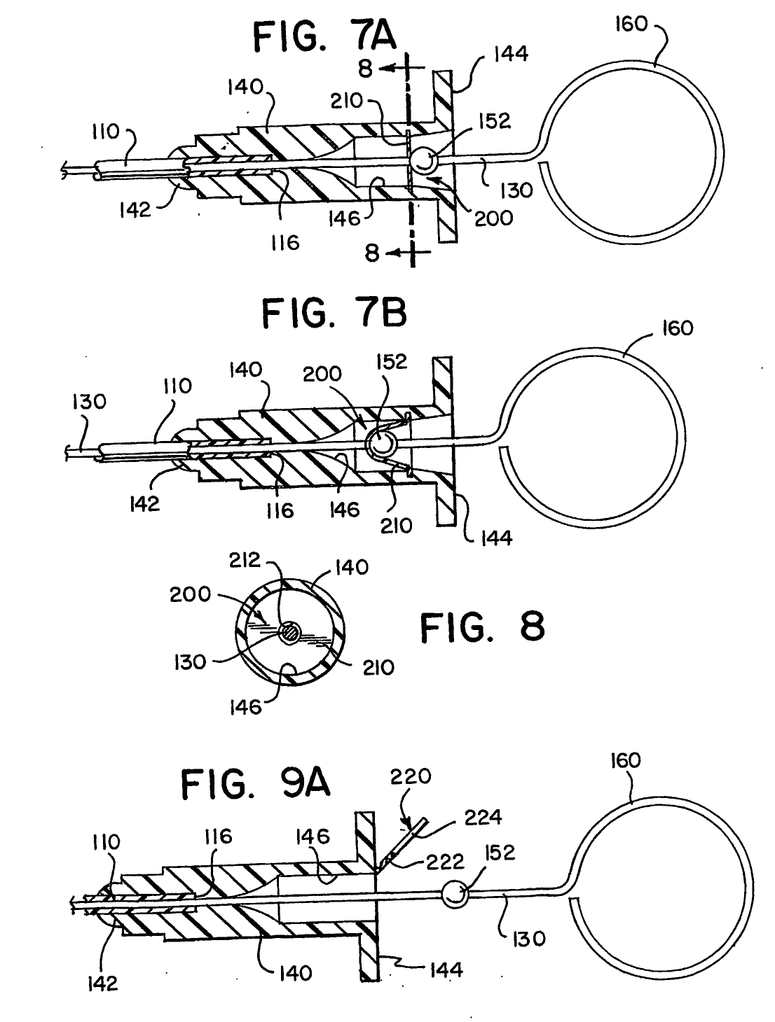

In one embodiment shown in FIGS. 7A, 7B and 8, the present dosing device 100 includes a stop mechanism 200 that is complementary to the interference element 152, the two of which serve to limit the degree of travel of the stylet 130 within the dosing guide tube 110. The stop mechanism 200 is specifically designed to create a selective interference with the interference element 152 and accordingly, can take any number of different forms. For example and according to one embodiment, a deformable stop 210 can be provided between the dosing guide tube 110 and the stylus 130 to prevent the stylus 130 from advancing within the dosing guide tube 110 to allow for easier loading of the solid dosage form and prevent accidental premature expulsion of the solid dosage form 121 from the dosage dispensing tip member 120. The illustrated deformable stop 210 is in the form of a deformable membrane that has a central opening 212 formed therein to permit the stylet 130 to freely travel therethrough; however, the opening 212 is undersized relative to the interference element 152 so as to prevent the interference element 152 from moving freely past the deformable membrane 210 prior to the actuation of the dosing device 100. More specifically, prior to the deformation of the membrane 210, the stylet 130 can not advance further axially within the guide tube 110 since the interference element 152 will abut against the deformable membrane 210 to prevent the interference element 152 from further entering the body member 140, thereby preventing the tip member 150 from contacting the dosing solid 121 in the reservoir. In other words, the tip member 150 does not extend beyond the first end 122 of the dosage dispensing tip member 120 prior to actuation of the dosing device 100 and therefore, the chance of contact with the animal's body itself is minimized.

The deformable membrane 210 can be formed of any number of materials. For example, the deformable membrane 210 can be formed as a thin deformable plastic membrane with the opening 212 formed therein. The membrane 210 is of sufficient integrity to prevent the interference element 152 from piercing the membrane 210 during normal storage and during positioning of the device 100 to the target site (travel of the device 100 down the esophagus); however, once a sufficient amount of force is applied by the interference element 152 against the deformable membrane 210, the membrane 210 deforms, thereby permitting the interference element 152 to advance further axially into the guide tube 110 resulting in the tip member 150 contacting the dosing solid 121. Many types of materials have the above memory characteristics and in one embodiment, the membrane 210 is made of a resilient synthetic material. One preferred type of material is a rubber material; however, other materials can be equally used.

The deformable membrane 210 has memory characteristics in that as soon as the force against the stylet 130 is removed, as by retraction of the stylet 130, the membrane 210 substantially returns to its original non-depressed condition.

In yet another embodiment and as will be appreciated by the reader, the deformable membrane 210 can be modified such that it is in the form of a rupturable membrane instead of a deformable membrane. More specifically, the area of the rupturable membrane, which can be formed of a thin plastic material or even a thin metal layer (e.g., aluminum), that is to contact the interference element can be of reduced thickness so that is represents a weakened region relative to the surrounding regions and therefore, it is more prone to rupture than the other regions when a force is applied thereto by the tip member 150.

As soon as the rupturable membrane breaks, the stylet 130 can continue to advance longitudinally within the bore 112 of the guide tube 110 so as to permit the tip member 150 to enter the reservoir 127 that contains the dosing solid 121 and make contact therewith for driving the dosing solid 121 out of the dosing dispensing tip member 120 once the device 100 is at the target site.

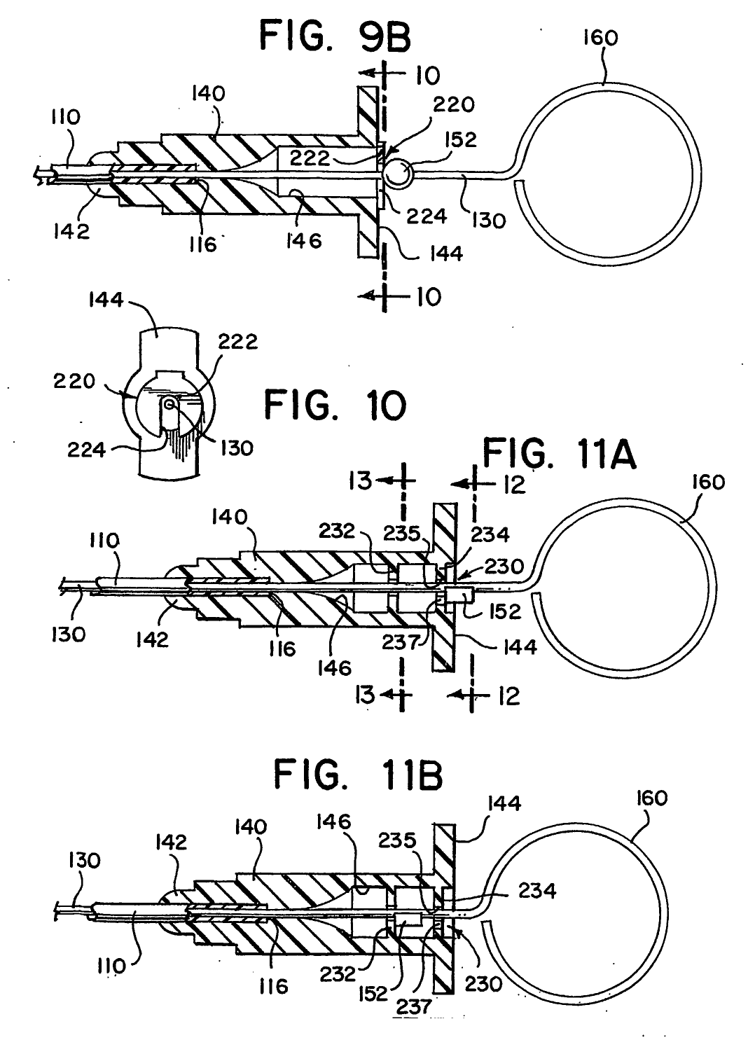

In yet another embodiment shown in FIGS. 9A, 9B and 10, an alternative stop mechanism 220 is provided. In this embodiment, the stop mechanism 220 includes a pivotable stop 222 that has a slot 224 to receive the stylet 130. The stop 222 defines the second end 144 of the body member 140 and is pivotably coupled to the remaining part of the body member 140 to which it is pivotally attached. The stop 222 pivots between a closed position where the interference element 152 is prevented from entering the body member 140, thereby restricting and limiting the axial advancement of the stylet 130 within the guide tube 110 and an open position, where the interference element 152 is permitted to enter the body member 140, thereby allowing the stylet 130 to be axially advanced such that the tip member 150 enters reservoir 127 and drives the dosing solid 121 out of the dosage dispensing tip member 120. For example, the stop 222 can be attached to the adjacent hollow body member 140 with a hinge or the like.

The slot 224 is preferably a radial slot that is closed at one end that is at or near the center of the stop 222 and is open at the outer circumferential edge of the stop 222. The width of the slot 224 is selected such that the stylet 130 can be received therein; however, the interference element 152 is sized such that it can not pass through the slot 224 while the stop 222 is in the closed position. Thus, in the closed position, the degree of axial travel of the stylet 130 in the guide tube 110 is limited.

To release the stylet 130 and permit it to advance axially within the guide tube 110 such that the tip member 150 advances into the reservoir 127 and contacts and drives the dosing solid 121 from the reservoir 127, the stop 222 is pivoted to the open position by simply grasping an edge of the stop 222 with one finger while holding the body member 140 and then apply a force against the edge so as to cause the stop 222 to pivot open. Once the stop 222 pivots open, the interference element 152 can travel into the body member 140, thereby permitting further axially travel of the stylet 130 such that the tip member 150 contacts and discharges the dosing solid 121. The design of the slot 224, and more particularly, its open nature at the circumferential edge, permits the stop 222 to pivot without disrupting the stylet 130 since the slot 224 accommodates the stylet 130 as it pivots open.

In yet another embodiment shown in FIGS. 11A, 11B, 12 and 13, an alternative stop mechanism 230 is provided and in combination with the interference element 152 resembles a lock and key arrangement. More specifically, the interference element 152 is the key element, while the body member 140 is constructed to act as a lock as described below. According to one exemplary embodiment, the lock part of the mechanism is formed of a pair of spaced first and second cross walls 232, 234 that extend across the bore 146 of the body member 140 and thus across the width of the body member 140 at or near the second end 144. Preferably, the second wall 234 in fact closes off the bore 146 at the second end 144 with the first wall 232 being formed more toward the first end 142 and in a parallel arrangement relative to the second wall 234.

It is the design of the first and second walls 232, 234 that acts as the stop (lock) member and selectively controls when and the degree of advancement of the stylet 130 in the following manner. As can be seen in the figures, the stylet 130 typically has a circular cross-section, although other shapes are equally possible, and a complementary outer opening 235 is formed through the second wall 234. Preferably, the outer opening 235 has an identical cross-sectional shape as the cross-sectional shape of the stylet 130, with the outer opening 235 having a larger diameter than the diameter of the stylet 130 to permit the stylet 130 to comfortable extend therethrough and be axially moved therein. The interference element 132, that acts as the key, is in the form of a projection that extends locally away from the arcuate outer surface of the stylet 130. For example, the projection 152 (key) can have a generally rectangular shape; however, any other shape is equally possible, such as a circle, square, triangle, etc.

The outer opening 235 is constructed and designed so that it can receive the projection 152 and therefore, the outer opening 235 includes a notch 237 that has a shape complementary to the shape of the projection 152 that extends radially outward from the circular opening that has a shape complementary to the stylet body 130. The outer opening 235 thus represents a key hole that only permits the key (projection) to be inserted when the two are in proper registration with one another. In other words, if the projection 152 is not properly aligned with the notch 237, the projection will strike the second wall 234 which acts as a stop since continued axial travel of the stylet 130 in the body member 140 and the guide tube 110 is prevented. This position is a pre-actuation position where the tip member 150 does not extend beyond the dosage dispensing tip member 120 and more particularly, the tip member 150 lies outside of the reservoir 127.

Conversely and as soon as the stylet 130 is rotated so as to place the projection 232 in alignment with the notch 237, the stylet 130 can be axially advanced towards the dosage dispensing tip member 120, thereby brining the tip member 150 into contact with the dosing solid 121. In other words, the registration between the two members acts according to a key-in-lock principal and permit the stylet a further degree of axial movement when actuation of the device 100 is desired when the dosage dispensing tip member 120 is at the target location.

The first wall 232 is similar but different from the second wall 234 in that the first wall 232 only includes a through opening 233 that has a cross-sectional shape to match the cross-sectional shape of the stylet 130. In other words, the first wall 232 does not have a notch extending radially outward therefrom and therefore, the projection 232 is not accommodated by the through opening 232. Thus, the stylet 130 can be advanced axially forward only until the projection 152 strikes the first wall 232. Since the first wall 232 can not accommodate the projection 152, the first wall 232 acts as a stop and prevents any further axial movement of the stylet 130 toward the dosage dispensing tip member 120.

It will be appreciated that the spacing between the first and second walls 232, 234 (e.g., the distance between these two walls 232, 234) controls the degree of travel of the stylet 130 after actuation of the device 100. More specifically, when the projection 152 strikes the first wall 232, the tip member 150 has reached its maximum travel point from and relative to the dosage dispensing tip member 120. Thus, this distance between the walls 232, 234 is selected such that the stylet 130 can not be advanced too far in the axial direction so as to unnecessarily strike the esophagus or other body part of the animal. The distance between the two is therefore carefully tailored so that when the projection 152 strikes the first wall 232, the tip member 150 has passed through and cleared the dosage dispensing tip member 150, thereby discharging all of the dosing solids 121 stored therein, and is at the end of its axial travel. The advantage of providing and constructing the first wall 232 to act as a stop is that it prevents the first end of the stylet 130 from advancing too far beyond the dosage dispensing tip member 120 and into contact with the esophagus or the like which it can damage. To retract the stylet 130 so as to reset the stylet 130 and permit additional loading of one or more dosing solids 121 into the reservoir 127, the user simply rotates the stylet 130 so as to align the projection 152 with the notch 237 and then pulls the stylet 130 away from the guide tube 110. As a result of the registration between the projection 152 and the notch 237, the projection 152 can clear the second wall 234 and be moved outside of the body member 140.

FIGS. 14A, 14B and 15 illustrate another embodiment that is similar to the embodiment of FIGS. 13A, 13B, and 14 in that a stop mechanism 240 is provided and generally follows the above key-in-lock principal. More specifically and according to the stop mechanism 240, a lock member similar to the one described above is provided and includes only one end wall 242 that is formed at or near the second end 144 of the body member 140. The end wall 242 closes off the body member 140 at or near the second end 144 thereof and includes a through opening 246 formed therein to provide communication and an entrance to the bore 146 of the body member 140.

The through opening 246 is preferably the same as the outer opening 235 of the previous embodiment in that it is constructed and designed so that it can receive the first projection 152 and therefore, the through opening 246 includes the notch 237 that has a shape complementary to the shape of the projection 152 that extends radially outward from the circular opening that has a shape complementary to the stylet body 130. The outer openings 235 thus represents a key hole that only permits the key (projection 152) to enter only when the two are in proper registration with one another.

In this embodiment, the stylet 130 also includes a second interference member (e.g., a second projection) 248 that is formed as part of the stylet 130 between the first projection 152 and the second end 134 of the stylet 130. The second interference element 248, acts as a stop, and preferably is in the form of a projection that extends locally away from the arcuate outer surface of the stylet 130 as does the first interference element 132. For example, the second projection 248 can have a generally rectangular shape; however, any other shape is equally possible, such as a circle, square, triangle, etc. The second projection 248 is radially and axially offset from the first projection 152. In other words, the two projections 152, 248 do not lie within the same plane along the length of the stylet 130 and therefore, the registration and passage of the first projection 152 through the notch 137 and further axial advancement of the stylet 130 does not result in the second projection 248 passing through the notch 137 but rather, the second projection 248 will strike the end wall 242. The striking of the second projection 248 against the end wall 242 acts as a stop since it prevents further axial advancement of the stylet 130 within the guide tube 110. The dimensions of the second projection 248 only need to be such that it can not pass through the circular cross-section through opening 246.

It will be appreciated that the spacing between the first and second projections 152, 248 (e.g., the distance between these two projections) controls the degree of travel of the stylet 130 after actuation of the device 100. More specifically, when the second projection 248 strikes the end wall 242, the tip member 150 has reached its maximum travel point from and relative to the dosage dispensing tip member 120. Thus, this distance between the first and second projections 152, 248 is selected such that the stylet 130 can not be advanced too far in the axial direction so as to unnecessarily strike the esophagus or other body part of the animal. The distance between the two is therefore carefully tailored so that when the projection 152 strikes the end wall 242, the tip member 150 has passed through and cleared the dosage dispensing tip member 150, thereby discharging all of the dosing solids 121 stored therein, and is at the end of its axial travel. The advantage of providing and constructing the end wall 242 and the second projection 248 to act as a stop is that it prevents the first end of the stylet 130 from advancing too far beyond the dosage dispensing tip member 120 and into contact with the esophagus or the like which it can damage.

In yet another embodiment illustrated in FIGS. 5A, 5B and 6, an alternate dosage dispensing tip member 260 is constructed such that it can hold one or more dosing solids 121 for sequential dispensing of the solids to the animal. For example, the reservoir 127 can be expanded to house more than one dosing solid such that features 250 that are formed as part of the tip member 260 serve to separately hold the one or more dosing solids 121. The features 250 that are part of the tip member 260 can be any number of different structures that serve to hold the dosing solids 121 in place and more preferably, they serve to hold the dosing solids 121 in place separate from one another. For example, the features 250 can be flexible ribs 252 formed along an inner surface that defines the bore that extends through the tip member 260 with these ribs 252 serving to partition the reservoir 127 into multiple compartments 254, with each compartment 254 holding one dosing solid 121. In the illustrated embodiment shown in FIGS. 5A and 5B, the features 250 are in the form of three ribs that are off set and spaced from one another so as to define 3 distinct compartments 254 that each holds a dosing solid. Each rib 252 can have any number of different shapes that range from one simple finger like projection to a flexible annular rib to a segmented annular rib. The flexible nature of the ribs 252 permits the loading and discharging of the dosing solids 121 without damaging the ribs 252 and in such away that the dosing tip member 260 can be used multiple times since, after deformation thereof to permit the dosing solid 121 to either be loaded or discharged, the ribs 252 return to their original state as a result of the resilient properties of the material from which the ribs 252 are formed.

To load the dosing solids 121 into the reservoir 127, the first dosing solid 121 is disposed into the first compartment at the first end 122 of the tip member 260 and then by action of an instrument or the like, the dosing solid is pushed passed the deformable first rib that is closest to end 122, thereby entering the second compartment. If only two dosing solids 121 are to be dispersed, then the second dosing solid 121 is inserted into the first compartment after the other dosing solid 121 has been placed into the second compartment. If three dosing solids 121 are being used, then after disposing the dosing solid 121 within the second compartment, the dosing solid 121 is then advanced into the third compartment that is furthest from the end 122 by using an instrument or the like. The dosing solid 121 is thus pushed past another deforming rib 252, that is closest to the second end 124 of the tip member 260. As soon as the dosing solid 121 is loaded into the third compartment, another dosing solid 121 is loaded and under action of the tool is advanced past the first deforming tab 252 until this second dosing solid 121 is placed into the second compartment past the deforming rib. This second dosing solid 121 is held in place between the two spaced ribs 252. Lastly, the third dosing solid is loaded into the first compartment. It will also be understood that each compartment can hold more than one dosing solid.

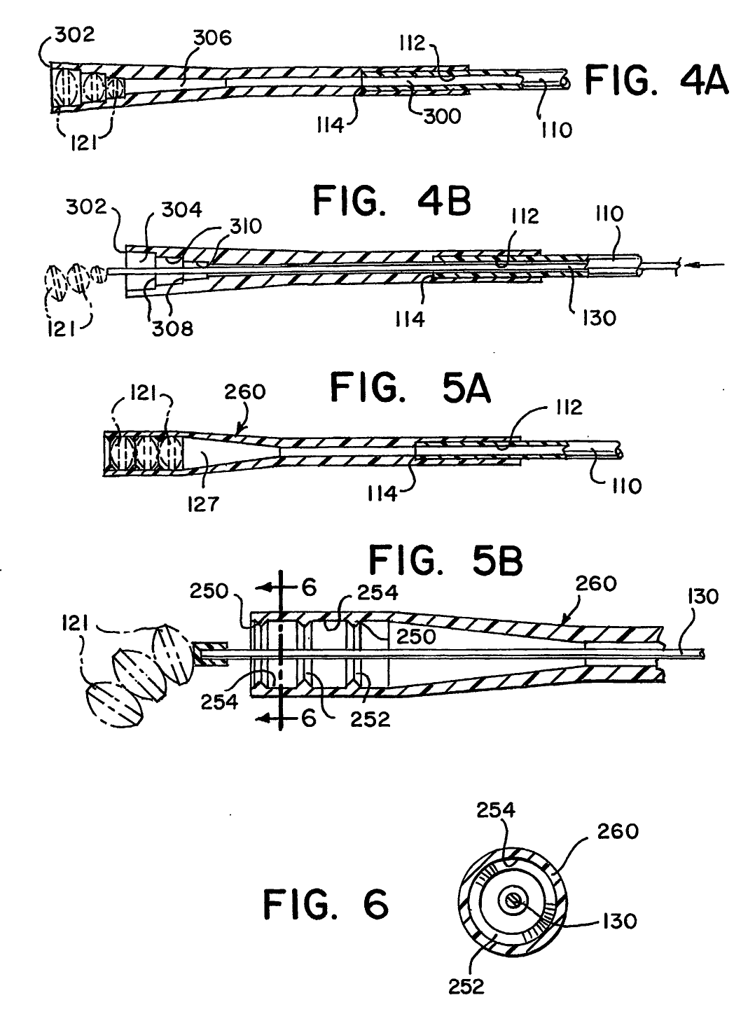

In FIGS. 4A-4B, a dosage dispensing tip member 300 according to another embodiment is illustrated and is constructed to hold more than one dosage solid 121. In this embodiment, a first end 302 that has a generally frustoconical shape and defines a dosage reservoir 304 that holds the dosing solids 121. A bore 306 that is formed through the tip member 300 has a stepped construction in the dosage reservoir 304 in that one or more shoulders 308 are formed in the dosage reservoir along an inner wall thereof. In the illustrated embodiment, two shoulders 308 are formed and define three separate compartments 310 that each holds one dosage solid 121. The shoulders 308 and the stepped construction of the dosage reservoir 304 serve to limit the diameter of the bore 306 in the reservoir 304, thereby permitting different sized dosage solids 121 to be received in selected compartments 310. For example and as shown in the illustrated embodiment, the innermost compartment 310 holds the smallest diameter dosage solid 121, while the outermost compartment 310 holds the largest diameter dosage solid 121, with the middle sized dosage solid 121 being in the middle. This construction permits multiple sized dosing solids 121 to be used and dispensed to the animal. It will be appreciated that in the situation where only two different sized dosing solids 121 are used, then all three of the compartments 310 are not used but rather, the two compartments 310 into which the dosage solids 121 frictionally fit and can be retained in are the ones that are used. For example, if one small and one large dosing solid 121 are used, then the innermost and the outermost compartments 310 are occupied, with the middle compartment 310 being empty. The dosing solids 121 are preferably frictionally fit into and held within the compartments 310.

In another embodiment of the invention the stylet 130 and/or the inside surface of the dosing guide tube 110 can be coated with an acceptable lubricant, such as K-Y® brand jelly (McNeil Consumer & Specialty Pharmaceuticals, Fort Washington, Pa.), or silicone.

In yet another embodiment of the present invention the dosing guide tube 110 can contain, at least in part, a radiopaque material so the tube can be easily visualized on radiograph.

In another embodiment of the present invention, the outer surface of the dosing guide tube 110 can be imbedded or coated with a chemical or pharmacologic agent. For example the outer surface of the dosing tube can be coated with an acceptable lubricant, such as K-Y® brand jelly or silicone. Alternatively the outer surface of the dosing guide tube 110 can be coated with a local anesthetic to reduce the gag reflex and discomfort of the gavage. Further, the outer surface of the dosing guide tube 110 can be coated with a steroid to reduce localized swelling from the gavage process. Additionally, the outer surface of the dosing guide tube 110 can be coated with a muscle tone modifying agent to reduce the tone of the esophageal sphincter. These tone modifying agents, but are not limited to peppermint, paralytic agents including anti-cholineric agents, benzodiazepines, anesthetics, and other muscle relaxers.

It is within the intended scope of the present invention that the inventive dosing apparatus can be used through any orifice, natural, surgical or traumic, including, but not limited to the mouth, nose, ears, vagina, urethra, rectum, eye sockets, gills, or blow-holes.

According to one application, a solid dosage form 121 is inserted into the flexible tip member 120 at the first end 114 of the dosing guide tube 110. Dosing guide tube 110 is inserted into the rat's mouth and carefully threaded down the rat's pharynx and esophagus about 8 cm to about 15 cm depending on the weight of the rat, preferably about 10 cm. The tablet is delivered into base of the esophagus and/or stomach by pushing the finger support 160 in the direction of the body member 140, thus advancing the stylet tip 150 through and beyond the flexible dosage dispensing tip member 120 pushing the solid dosage form 121, or anything else one may want to delivery to a rat's esophagus or stomach, out of dosage dispensing tip member 120 and into the rat's esophagus and/or stomach.

In a conscious rat, the solid dosage form 121 can be delivered in the posterior pharynx, or anywhere in the esophagus and the solid dosage form will be swallowed into the stomach. In an anesthetized or decerebrate rat, it may be more important to deliver the dose into the stomach or at least to the distal esophagus in or about the lower esophageal sphincter. Delivery of this nature can be facilitated by a reduction in the sphincter tone, as may be inherent in the anesthetic of decerebrate condition, or with use of other means.

Turning now to the solid dosage form 121, the solid dosage form for dosing rodents is described herein. A solid dosage form 121 can be made by the following method: a pharmaceutically or chemically active agent is gradually added and blended with the carrier. Upper punch, lower punch and die of a manual pellet press with a caplet shape model, such as a Carver 4350 manual pellet press with a caplet shape model sold by Natoli Engineering Company, Inc. are treated with an excipient, such as a lubricants, such as magnesium stearate. The mixed powder was fed into the die and a mini cylindrical shape tablet is made at about 1000 PSI bar pressure. The resultant solid dosage form has a diameter of about 0.5 mm to about 3.0 mm, preferably about 2.0 to about 20.5 mm, more preferably about 20.5 mm.

A capsule may be made as follows. A gelatin capsule, such as is sold by Torpac, Inc. can be filled with a liquid using a syringe, funnel or by any other means known in the art. A gelatin capsule, such as is sold by Torpac, Inc. can be filled with a solid using a syringe, funnel, pipette tip, spatula (including a flattened paper clip tip) or by any other means known in the art.

The solid dosage forms 121 of the present invention may include a delivery agent and an active agent. These solid dosage forms may be used to deliver various active agents through various biological, chemical, and physical barriers and are particularly suited for delivering active agents which are subject to environmental degradation.

Delivery Agents

The delivery agent may be any of those described in U.S. Pat. Nos. 5,650,386 and 5,866,536 and International Publication Nos. WO94/23767, WO95/11690, W095/28920, O95/28838, W096/10396, W096/09813, WO96/12473, WO96/12475, WO96/30036, WO96/33699, WO97/31938, WO97/36480, WO98/25589, W098/34632, W098/49135, WO99/16427, WO00/06534, WO00/07979, WO00/40203, WO00/46182, WO00/47188, WO00/48589, WO00/50386, WO00/59863, WO00/59480, WO01/32130, WO01/32596, WO01/34114, WO01/44199, WO01/51454, WO01/70219, WO01/92206, WO02/02509, WO02/15959, WO02/16309, WO02/20466, WO02/19969, WO02/070438, WO03/026582, and WO02/100338 and WO03/045306 all of which are hereby incorporated by reference. Preferred delivery agents include Sodium N-[4-(4-chloro-2-hydroxybenzoyl)amino]butyrate (4-CNAB), N-(8-[2-hydroxybenzoyl]amino)caprylic acid and N-(10-[2-hydroxybenzoyl]amino)decanoic acid and salts thereof, and solvates and hydrates thereof.

The delivery agent compounds may be in the form of the carboxylic acid or salts thereof. The salts may be mono- or multi-valent salts, such as monosodium salts and di-sodium salts. The salts may also be solvates, including ethanol solvates, and hydrates. The delivery agent compounds may be in the form of the free base or salts thereof.

Active Agents

Active agents suitable for use in the present invention include biologically active agents and chemically active agents, including, but not limited to, pesticides, pharmacological agents, and therapeutic agents.

For example, biologically or chemically active agents suitable for use in the present invention include, but are not limited to, proteins; polypeptides; peptides; hormones; polysaccharides, muco-polysaccharides and particularly mixtures of muco-polysaccharides; carbohydrates; lipids; small polar organic molecules (i.e. polar organic molecules having a molecular weight of 500 daltons or less); other organic compounds; and particularly compounds which by themselves do not pass (or which pass only a fraction of the administered dose) through the gastro-intestinal mucosa and/or are susceptible to chemical cleavage by acids and enzymes in the gastro-intestinal tract; or any combination thereof.

Further examples include, but are not limited to, the following, including synthetic, natural or recombinant sources thereof: growth hormones, including human growth hormones (hGH), recombinant human growth hormones (rhGH), bovine growth (hGH), recombinant human growth hormones (rhGH), bovine growth hormones, and porcine growth hormones; growth hormone-releasing hormones; growth hormone releasing factor (e.g., GRF analog g); PYY; Anthrax; Pestis; interferons, including α, β and γ; interleukin-1; interleukin-2; insulin, including porcine, bovine, human, and human recombinant, optionally having counter ions including zinc, sodium, calcium and ammonium; insulin-like growth factor, including IGF-1; heparin, including unfractionated heparin, heparinoids, dermatans, chondroitins, low molecular weight heparin, very low molecular weight heparin and ultra low molecular weight heparin; calcitonin, including salmon, eel, porcine and human; erythropoietin; atrial naturetic factor; antigens; monoclonal antibodies; somatostatin; protease inhibitors; adrenocorticotropin, gonadotropin releasing hormone; oxytocin; leutinizing-hormone-releasing-hormone; follicle stimulating hormone; glucocerebrosidase; thrombopoietin; filgrastim; prostaglandins; cyclosporin; vasopressin; cromolyn sodium (sodium or disodium chromoglycate); vancomycin; desferrioxamine (DFO); bisphosphonates, including alendronate, tiludronate, etidronate, clodronate, pamidronate, olpadronate, and incadronate; parathyroid hormone (PTH), including its fragments; anti-migraine agents such as BIBN-4096BS and other calcitonin gene-related proteins antagonists; antimicrobials, including antibiotics (include gram-positive acting, bacteriocidal, lipopeptidal and cyclic peptidal antibiotics, including daptomycin), anti-bacterials and anti-fungal agents; vitamins; analogs, fragments, mimetics or polyethylene glycol (PEG)-modified derivatives of these compounds; or any combination thereof. Non-limiting examples of antibiotics and analogs thereof.

### EXAMPLE 1

### Solid Dose Manufacture

About 0.125 mg/tablet (about 0.5 mg/kg) of Insulin was gradually added and blended with about 10 mg/tablet (about 40 mg/kg) Sodium N-[4-(4-chloro-2-hydroxybenzoyl)amino]butyrate (4-CNAB). Upper punch, lower punch and die of Carver 4350 manual pellet press with a Caplet shape model sold by Natoli Engineering Company, Inc. were treated with magnesium stearate (0.1%). 10.125 mg of mixed powder was fed into the die and a mini bead shape tablet was made at about 1000 PSI bar pressure. The resulting solid dosage form is about 2.5 mm diameter.

### EXAMPLE 2

### Dosing Procedure in Rats

A Sprague Dawley rat (230˜250 g) was anesthized by standard CO₂ inhalation technique for about 10 to 30 seconds resulting in an anesthesized state for about less then one minute, preferably about 10 to about 30 seconds.

An oral dosing guide tube 110 was used. The solid dosage form 121 was inserted into the dosage dispensing tip member 120 at the first end 114 of the dosing guide tube 110. The dosing guide tube 110 was inserted into the rat's mouth and carefully threaded down the rats pharynx and esophagus about 8 cm to about 15 cm depending on the weight of the rat (about 200 g to a about 250 g thread down about 10 cm; for about 250 g to about 300 g thread down about 11 cm; for about 300 g to a about 350 g, thread down about 12 cm; for about 350 g to a about 400 g, thread down about 13 cm; for about 400 g to a about 450 g, thread down about 14 cm; in this case for about 230 g to a about 250 g, thread down about 10 cm.

The solid dosage form 121 was delivered into the distal esophagus and/or stomach by pushing the finger support 160 in the direction of dispensing tip member 120, thus advancing stylet tip member 150 through dosage dispensing tip member 120 pushing the solid dosage form 121 out of dosage dispensing tip member 120 and into the rat's distal esophagus and/or stomach.

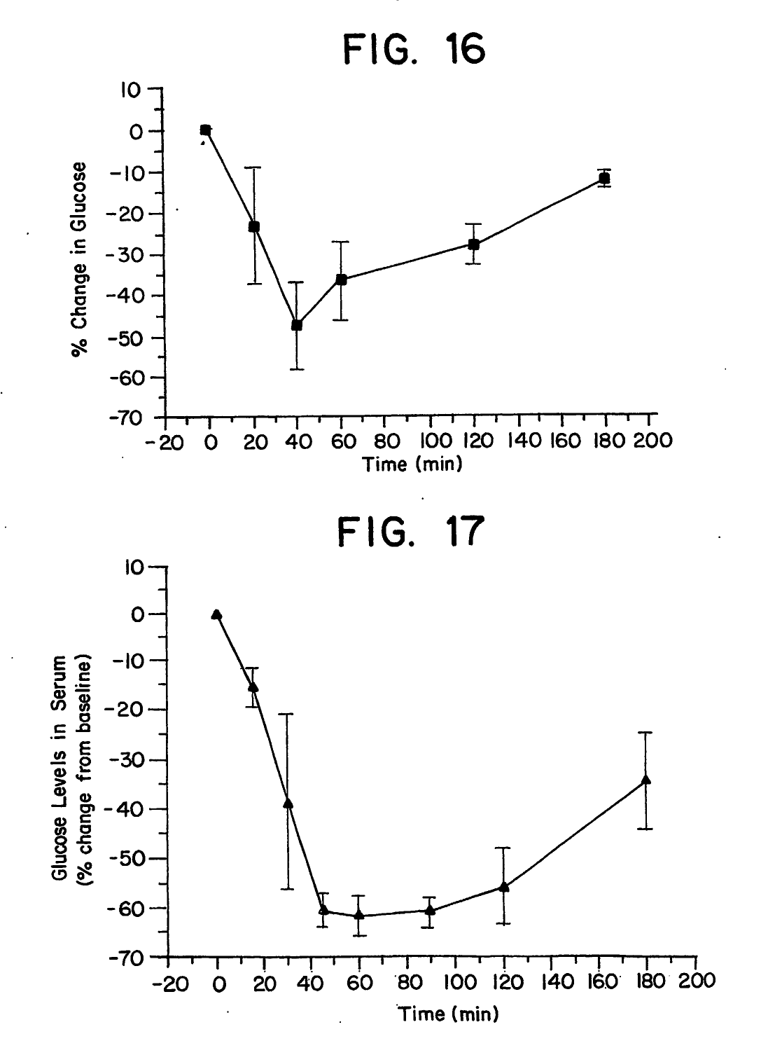

Blood samples were collected via standard procedures. For instance, blood samples were collected via tail clipping method at designated time points (0, 20, 40, 60, 120, 180 minute) to measure glucose levels in assessing insulin formulations. Each rat's tail was cut at the tip (about 2 mm of the tail). The first drop of blood from the animal's tail was not used for glucose readings. A fresh drop of blood from the tip of each rat's tail was placed on the tip of the test strip. The test strip used, such as the Onetouch Ultra by Lifescan of Milpitas, Calif., to determine the level of glucose in blood samples were only exposed to light as needed. All test strips were stored individually in closed vials except during use. After baseline measurements, the rats were orally dosed. The percent change in glucose levels from baseline is shown in FIG. 16.

Optionally, blood can be collected from the tail artery with use of a general anesthetic such as Ketamine or Thorazine or retro-orbitally using CO₂ anesthesia. Optionally, other tests for Glucose and insulin can be used including serum insulin, c-peptide levels, Hb_{Alc}. At two minutes post dosing, it was found by necropsy examination that all solid dosage forms were in the stomach of the subject rats.

### EXAMPLE 3

### Solid Dose Manufacture with SNAC

About 0.125 mg/tablet (about 0.5 mg/kg) of Insulin was gradually added and blended with about 5 mg/tablet (about 20 mg/kg) Sodium N-(8-[2-hydroxybenzoyl]amino)caprylate (SNAC). Upper punch, lower punch and die of Carver 4350 manual pellet press with a Caplet shape model sold by Natoli Engineering Company, Inc. were treated with magnesium stearate (0.1%). 10.125 mg of mixed powder was fed into the die and a mini bead shape tablet was made at about 1000 PSI bar pressure. The resulting solid dosage form is about 2.5 mm diameter.

### EXAMPLE 4

### Dosing Procedure in Rats of Solid Dosage Form of Example 3

A Sprague Dawley rat (230˜250 g) was anesthized by standard CO₂ inhalation technique for about 10 to 30 seconds resulting in an anesthesized state for about less then one minute, preferably about 10 to about 30 seconds. An oral dosing guide tube 110 was used. The solid dosage form was inserted into the dosage dispensing tip member 120 at the first end 114 of the dosing guide tube 110. The dosing guide tube 110 was inserted into the rat's mouth and carefully threaded down the rats pharynx and esophagus about 8 cm to about 15 cm depending on the weight of the rat in this example for a rat of about 230 g to about 250 g, thread down about 10 cm.

The solid dosage form was delivered into the distal esophagus and/or stomach by pushing the finger support 160 in the direction of dispensing tip member 120, thus advancing stylet tip member 150 through dosage dispensing tip member 120 pushing the solid dosage form 121 out of flexible dosage dispensing tip member 120 and into the rat's distal esophagus and/or stomach.

Blood samples were collected via standard procedures. For instance, blood samples were collected via tail clipping method at designated time points (0, 20, 40, 60, 120, 180 minute) to measure glucose levels in assessing insulin formulations. Each rat's tail was cut at the tip (about 2 mm of the tail). The first drop of blood from the animal's tail was not used for glucose readings. A fresh drop of blood from the tip of each rat's tail was placed on the tip of the test strip. The test strip used, such as the Onetouch Ultra by Lifescan of Milpitas, Calif., to determine the level of glucose in blood samples were only exposed to light as needed. All test strips were stored individually in closed vials except during use. After baseline measurements, the rats were orally dosed. The percent change in glucose levels from baseline is reported in FIG. 17.

In the description, reference is made to the accompanying drawings, which form a part hereof, and which illustrate examples of the invention. Such examples, however, are not exhaustive of the various embodiments of the invention, and therefore, reference is made to the claims which follow the description for determining the scope of the invention. While illustrative embodiments of the invention have been described, it is, of course, understood that various modifications of the invention will be obvious to those of ordinary skill in the art. Such modifications are within the spirit and scope of the invention which is limited and defined by the appended claims. Accordingly, the specification is to be regarded in an illustrative manner rather than a restrictive sense.

While we have hereinbefore described a number of embodiments of this invention, it is apparent that our basic constructions can be altered to provide other embodiments that utilize the processes and compositions of this invention. Therefore, it will be appreciated that the scope of this invention is to be defined by the claims appended hereto rather than by the specific embodiments that have been presented hereinbefore by way of example.

What is claimed is:

1. An apparatus for selectively dosing a solid comprising:
a flexible guide tube that includes a first end and an opposing second end and has a bore formed therethrough from the first end to the second end;
a dispensing tip disposed at the first end of the guide tube and including a bore formed completely therethrough and defining a reservoir for holding one or more dosing solids until actuation of the apparatus; and
a stylet that is dimensioned so as to be slideably received within and through the bore of the flexible guide tube, the stylet having a first end and an opposing second end, with the first end of the stylet being for contact with the one or more dosing solids to cause discharge thereof through the dispensing tip.

2. The apparatus of claim 1, wherein the flexible guide tube is formed of a plastic material that permits the guide tube to be bendable.

3. The apparatus of claim 1, wherein the dispensing tip is a separate part from the guide tube and is therefore interchangeable with the guide tube and is formed of a material that permits the dispensing tip to be flexible.

4. The apparatus of claim 3, wherein the dispensing tip is formed of a plastic material and is constructed to be bendable.

5. The apparatus of claim 3, wherein the dispensing tip has a first end and a second end that is removably coupled to the first end of the guide tube.

6. The apparatus of claim 5, wherein the first end of the guide tube is frictionally fit and retained within the bore of the dispensing tip such that the bore of the dispensing tip is axially aligned with the bore of the guide tube to permit the stylet to slidingly travel therebetween.

7. The apparatus of claim 1, wherein the reservoir is formed in a frustoconical shaped portion of the dispensing tip and a length of the bore that extends through and defines in part the reservoir has a tapered construction.

8. The apparatus of claim 1, wherein the dispensing tip and the guide tube are a single integral part molded in-situ.

9. The apparatus of claim 1, further including:
a holder securely coupled to the second end of the guide tube, the holder including a bore formed therethrough from a first end to a second end such that the bore of the holder is axially aligned with the bore of the guide tube to permit axial travel of the stylet therebetween; and
a stop mechanism associated with the holder for selectively limiting the axial travel of the stylet within the guide tube.

10. The apparatus of claim 9, wherein the stylet includes a stop feature formed along a length thereof and the stop mechanism comprises a rupturable membrane disposed across the bore near or at the second end of the holder away from the guide tube, wherein prior to actuation, when the stop feature is external to or in an abutting relationship with the rupturable membrane, the first end of the stylet does not reach the dosing solid, which therefore remains securely contained in the reservoir.

11. The apparatus of claim 10, wherein the stop feature comprises an enlarged section of the stylet and actuation of the apparatus entails forcibly rupturing the membrane with the stop feature as the stylet is axially driven, thereby permitting the stylet to advance in the guide tube and the dispensing tip such that the first end of the stylet contacts, frees and discharges the dosing solid from the dispensing tip.

12. The apparatus of claim 9, wherein the stylet includes a stop feature formed along a length thereof and the stop mechanism comprises a deformable membrane disposed across the bore near or at the second end of the holder away from the guide tube, wherein prior to actuation, when the stop feature is external to or in an abutting relationship with the rupturable membrane, the first end of the stylet does not reach the dosing solid, which therefore remains securely contained in the reservoir.

13. The apparatus of claim 12, wherein actuation of the apparatus causes the deformation of the membrane by means of contact with the stop feature, which is in the form of an enlarged section of the stylet, as the stylet is axially driven, thereby permitting the stylet to advance in the guide tube and the dispensing tip such that the first end of the stylet contacts, frees and discharges the dosing solid from the dispensing tip, the membrane having memory such that once.

14. The apparatus of claim 13, wherein the deformable membrane is formed of a rubber material.

15. The apparatus of claim 9, wherein the bore formed within the holder has a tapered section and the stylet has a stop feature formed along a length thereof for limiting the axial travel of the stylet as a result of the stop feature having a lesser diameter than a diameter of the tapered section but a greater diameter than a diameter of a remaining length of the bore.

16. The apparatus of claim 9, wherein the stylet includes a stop feature formed along a length thereof and the stop mechanism comprises a hinged member that is pivotable coupled to the holder, the hinged member including a slot formed therein that is open at a perimeter edge to receive the stylet and permit axially movement thereof, the stop feature having greater dimensions than the slot so as to restrict axial movement of the stylet unless the hinged member pivots open, thereby permitting passage of the stop feature and further axial travel of the stylet.

17. The apparatus of claim 9, wherein the stylet includes a stop feature formed along a length thereof and the stop mechanism comprises a lock and key arrangement with the stop feature acting as the key and the stop mechanism serving as the lock that permits axial travel of the stylet only when the lock and key are in proper registration.

18. The apparatus of claim 17, wherein the stop mechanism comprises first and second spaced walls that extend across the bore of the holder, the first wall being an inner wall closer to the guide tube, each of the first and second walls including a through opening formed therethrough that is complementary to and permits axial travel of the stylet therethrough, wherein only the second wall includes a notch that opens into and extends radially from the through opening, the notch being complementary to and receiving the stop feature when registration exists therebetween so as to permit the stylet to advance in the guide tube and the dispensing tip such that the first end of the stylet contacts, frees and discharges the dosing solid from the dispensing tip.

19. The apparatus of claim 18, wherein the stop feature is in the form of a protrusion formed along and extending radial from the stylet, the protrusion having greater dimensions that a diameter of the through opening in the first wall so as to restrict further axial movement of the stylet, the notch being a slot formed radially from the through opening.

20. The apparatus of claim 18, wherein a distance between the first and second walls is selected such that when the stop feature contacts and is stopped by the second wall, the first end of the stylet extends slightly beyond the dosage dispensing tip member.

21. The apparatus of claim 17, wherein the stop feature is in the form of a first projection that extends radially from the stylet and a second projection that extends radially from the stylet, the first and second projections being radially offset from one another, the first projection being closer the first end of the stylet and wherein the stop mechanism being a wall that extends across the bore of the holder at or near the second end thereof, the wall having a through opening formed therein and a notch that opens into and extends radially from the through opening, the notch being complementary to and receiving the first projection when the two are in registration so as to permit the stylet to advance in the guide tube and the dispensing tip such that the first end of the stylet contacts, frees and discharges the dosing solid and then the second projection contacts the wall at a location other than where the notch is formed so as to stop the axial movement of the stylet.

22. The apparatus of claim 21, wherein a distance between the first and second projections is selected such that when the second projection contacts and is stopped by the wall, the first end of the stylet extends slightly beyond the dosage dispensing tip member.

23. An apparatus for selectively dosing a solid comprising:
a flexible guide tube that includes a first end and an opposing second end and has a bore formed therethrough from the first end to the second end;
a flexible dispensing tip being interchangeably and removably coupled to the first end of the guide tube and including a bore formed completely therethrough and defining a reservoir for holding one or more dosing solids until actuation of the apparatus;
a stylet that is dimensioned so as to be slideably received within and through the bore of the flexible guide tube, the stylet having a first end and an opposing second end, with the first end of the stylet being for contact with the one or more dosing solids to cause discharge thereof through the dispensing tip a holder provided at the second end of the guide tube, the holder including a bore formed therethrough from a first end to a second end such that the bore of the holder is axially aligned with the bore of the guide tube to permit axial travel of the stylet therebetween; and
a lock and key mechanism associated with the stylet and the holder for selectively limiting the axial travel of the stylet within the guide tube, the stylet having a stop feature formed along a length thereof that serves as a key feature, while the holder has a lock feature that receives the key only when the two are in proper registration to thereby permit the axial travel of the stylet.

24. The apparatus of claim 23, wherein the guide tube is formed of a plastic and is bendable along an entire length thereof.

25. The apparatus of claim 23, wherein the guide tube contains a radiopaque material.

26. The apparatus of claim 23, wherein an outer surface of the guide tube includes a layer of material selected from the group consisting of: a lubricant, a steroid, and muscle tone modifying agent.

27. A dosing kit containing a number of parts that assemble to form an apparatus for selectively dosing a solid, the kit comprising:
a flexible guide tube that includes a first end and an opposing second end and has a bore formed therethrough from the first end to the second end;
a plurality of flexible dispensing tips that are constructed to be interchangeably and removably coupled to the flexible guide tube, each dispensing tip being constructed to be disposed at the first end of the guide tube and including a bore formed completely therethrough and, wherein one section of the bore defines a reservoir for holding one or more dosing solids until actuation of the apparatus; and
a stylet that is dimensioned so as to be slideably received within and through the bore of the flexible guide tube, the stylet having a first end and an opposing second end, with the first end of the stylet being for contact with the one or more dosing solids to cause discharge thereof through the dispensing tip;
wherein each dispensing tip differs with respect to at least one parameter selected from the group consisting of: a number of dosing solids that can be contained and held in the reservoir thereof, a size of the dosing solid that can be contained and held in the reservoir and whether the reservoir can hold multiple sized dosing solids.

28. The kit of claim 27, wherein a first end of the guide tube is frictionally retained within the bore of the dispensing tip so as to permit it to be slideably removed therefrom.

29. The kit of claim 27, wherein one dispensing tip includes a plurality of compartments defined therein for separately holding a plurality of dosing solids, the compartments being defined in part by deformable ribs formed within the bore of the dispensing tip.

30. The kit of claim 27, wherein one dispensing tip has an inner stepped construction formed in the reservoir section of the bore, the inner stepped construction defining a plurality of shoulders that define individual compartments for holding plural dosing solids.

31. The kit of claim 30, wherein a diameter of one shoulder is different than a diameter of another shoulder so as to permit different sized dosing solids to be held and retained in the reservoir.

32. The kit of claim 27, further including:
a holder that is a separate part relative to the guide tube and is securely coupled to the second end of the guide tube, the holder including a bore formed therethrough from a first end to a second end such that the bore of the holder is axially aligned with the bore of the guide tube to permit axial travel of the stylet therebetween; and
a stop mechanism associated with the holder for selectively limiting the axial travel of the stylet within the guide tube.

33. The kit of claim 32, wherein the stylet includes a stop feature formed along a length thereof and the stop mechanism comprises a lock and key arrangement with the stop feature acting as the key and the stop mechanism serving as the lock that permits axial travel of the stylet only when the lock and key are in proper registration.

34. The kit of claim 33, wherein the stop mechanism comprises first and second spaced walls that extend across the bore of the holder, the first wall being an inner wall closer to the guide tube, each of the first and second walls including a through opening formed therethrough that is complementary to and permits axial travel of the stylet therethrough, wherein only the second wall includes a notch that opens into and extends radially from the through opening, the notch being complementary to and receiving the stop feature when registration exists therebetween so as to permit the stylet to advance in the guide tube and the dispensing tip such that the first end of the stylet contacts, frees and discharges the dosing solid from the dispensing tip.

35. The kit of claim 34, wherein the stop feature is in the form of a protrusion formed along and extending radial from the stylet, the protrusion having greater dimensions that a diameter of the through opening in the first wall so as to restrict further axial movement of the stylet, the notch being a slot formed radially from the through opening.

36. The kit of claim 34, wherein a distance between the first and second walls is selected such that when the stop feature contacts and is stopped by the second wall, the first end of the stylet extends slightly beyond the dosage dispensing tip member.

37. The kit of claim 34, wherein the stop feature is in the form of a first projection that extends radially from the stylet and a second projection that extends radially from the stylet, the first and second projections being radially offset from one another, the first projection being closer the first end of the stylet and wherein the stop mechanism being a wall that extends across the bore of the holder at or near the second end thereof, the wall having a through opening formed therein and a notch that opens into and extends radially from the through opening, the notch being complementary to and receiving the first projection when the two are in registration so as to permit the stylet to advance in the guide tube and the dispensing tip such that the first end of the stylet contacts, frees and discharges the dosing solid and then the second projection contacts the wall at a location other than where the notch is formed so as to stop the axial movement of the stylet.

38. The kit of claim 37, wherein a distance between the first and second projections is selected such that when the second projection contacts and is stopped by the wall, the first end of the stylet extends slightly beyond the dosage dispensing tip member.

39. A method of dosing a solid to a subject comprising the steps of:
providing the apparatus of claim 1;
inserting the apparatus into an orifice associated with the subject;
axially moving the stylet within the guide tube so as to cause displacement of the dosing solid from the reservoir and out of the dispensing tip to the patient.
